(19) United States
(12) Patent Application Publication
(10) Pub. No.: US 2014/0277493 A1
(43) Pub. Date: Sep. 18, 2014

(54) Implant Instrument Interconnection

(71) Applicant: Osteomed LLC, Addison, TX (US)

(72) Inventors: Jason M. Glad, Nibley, UT (US); Andrew R. Fauth, River Heights, UT (US); Charles R. Forton, Frisco, TX (US)

(73) Assignee: Osteomed LLC, Addison, TX (US)

(21) Appl. No.: 14/041,934

(22) Filed: Sep. 30, 2013

Related U.S. Application Data
(60) Provisional application No. 61/782,036, filed on Mar. 14, 2013.

(51) Int. Cl. A61F 2/46; A61F 2/44

(52) U.S. Cl. CPC A61F 2/4611; A61F 2/442
USPC 623/17.16

(57) ABSTRACT

An inserter instrument and an implant device are provided. An interconnection between an implant and an instrument may include a threaded connection and a gripping connection. Motion of the gripping connection is coordinated with motion of the threaded connection. One implant includes a threaded hole and an adjacent angled pocket, both of which extend into the implant through an instrument-facing surface. One instrument includes a threaded rod and an adjacent pivoting finger, both of which protrude from an end face of the instrument. When the implant is connected to this instrument, the instrument-facing surface abuts the end face, the threaded rod engages the threaded hole, and the finger extends into the pocket along an angle of the pocket.

### CROSS-REFERENCE TO RELATED APPLICATIONS

The present application claims priority to co-pending U.S. Provisional Patent Application No. 61/782,036, entitled “IMPLANT INSTRUMENTATION INTERCONNECTION,” filed Mar. 14, 2013, the disclosure of which is hereby incorporated herein by reference in its entirety.

### TECHNICAL FIELD

The present disclosure relates to interconnections between implants and instruments. While the present disclosure is made in the context of a spinal intervertebral spacer implant and an inserter instrument, the concepts embodied herein are applicable to other implant/instrument interconnections.

### BACKGROUND

Many different types of surgical tools are utilized to insert device implants into a human body. One implant that generally is placed using an inserter device is a spinal intervertebral spacer. Such a spacer is placed between two vertebrae and used to facilitate fusion of the respective vertebrae. Placing this device requires the use of different forces between a connection point of an insertion tool and the device, e.g. pushing, pulling, and prying, etc., due to the anatomy of the body. Accordingly, an inserter must be able to withstand these forces without losing the device or improperly placing the device.

One inserter device which is currently utilized may be referred to as a threaded inserter device. A threaded inserter device generally has a threaded extension that threads onto the implant device to secure the device during insertion and is configured to allow a surgeon to unthread the implant device from the extension after insertion. While such devices have been useful, problems may still occur due with respect to the connection between the inserter device and the implant device. For example, when exerting force on a connected implant device, rotational forces can be present that can cause the implant to be misaligned with the insertion space, and in some cases, can cause the implant device to back off of the insertion device. Additionally, current threaded devices are not the ideal design to account for torsional or pulling forces.

### BRIEF SUMMARY

The present application provides for systems, devices and methods which implement a connection between an inserter device and an implant that provides for both a primary connection and a secondary connection. In one example, the inserter device may include a threaded portion configured to connect to an implant device as well as one or more finger portions that provide for a gripping connection. The finger portions may be configured to reduce or prohibit rotation of the implant device. Further, the finger portions may be set at an angle with respect to the threaded portion to assist in mitigating pulling forces. In some embodiments, the finger portions may be configured to grip and release in coordination with the primary connection device.

In one embodiment an implant device may be provided that is configured to receive a primary and secondary connection from an inserter device. The primary connection configuration may include a threaded aperture which receives a threaded extension. Further, the secondary connection configuration may include one or more apertures configured to accept a gripping mechanism. Such apertures may be set at angles corresponding to the gripping mechanisms.

The foregoing has outlined rather broadly the features and technical advantages of the present invention in order that the detailed description that follows may be better understood. Additional features and advantages will be described hereinafter which form the subject of the claims. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of embodiments described herein, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present embodiments.

### BRIEF DESCRIPTION OF THE DRAWINGS

While exemplary embodiments of the present technology have been shown and described in detail below, it will be clear to the person skilled in the art that changes and modifications may be made without departing from its scope. As such, that which is set forth in the following description and accompanying drawings is offered by way of illustration only and not as a limitation. The actual scope of the invention is intended to be defined by the following claims, along with the full range of equivalents to which such claims are entitled.

In addition, one of ordinary skill in the art will appreciate upon reading and understanding this disclosure that other variations for the technology described herein can be included within the scope of the present technology.

In the following Detailed Description, various features are grouped together in several embodiments for the purpose of streamlining the disclosure. This method of disclosure is not to be interpreted as reflecting an intention that exemplary embodiments of the technology require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

Identical reference numerals do not necessarily indicate an identical structure. Rather, the same reference numeral may be used to indicate a similar feature or a feature with similar functionality. Not every feature of each embodiment is labeled in every figure in which that embodiment appears, in order to keep the figures clear. Similar reference numbers (e.g., those that are identical except for the first numeral) are used to indicate similar features in different embodiments.

FIG. 1 is an isometric view of an instrument connected to an implant;

FIG. 2 is an isometric detail view of a portion of the instrument of FIG. 1;

FIG. 3A is an end view of the instrument and implant of FIG. 1, looking at the implant end; and FIG. 3B is a cross sectional detail view of a portion of the instrument of FIG. 3A, taken along section line B-B of FIG. 3A;

FIG. 4 is an exploded top view of a portion of the instrument and implant of FIG. 1;

FIG. 5A is a side view of the instrument and implant of FIG. 1; and FIG. 5B is a cross sectional detail view of a portion of the instrument and implant of FIG. 5A, taken along section line E-E of FIG. 5A, the instrument in an open state;

FIG. 6A is a side view of the instrument and implant of FIG. 1; and FIG. 6B is a cross sectional detail view of a portion of the instrument and implant of FIG. 6A, taken along section line F-F of FIG. 6A, the instrument in a locked state;

FIG. 7A is an isometric view of the implant of FIG. 1; FIG. 7B is a top view of the implant of FIG. 1; FIG. 7C is a left view of the implant of FIG. 1; FIG. 7D is a front view of the implant of FIG. 1; and FIG. 7E is a right view of the implant of FIG. 1;

FIG. 8 is an isometric view of a portion of the instrument of FIG. 1, the instrument in an open state; and

FIG. 9 is an isometric view of a portion of the instrument of FIG. 1, the instrument in a locked state.

### DETAILED DESCRIPTION

Standard medical planes of reference and descriptive terminology are employed in this specification. A sagittal plane divides a body into right and left portions. A mid-sagittal plane divides the body into equal right and left halves. A coronal plane divides a body into anterior and posterior portions. A transverse plane divides a body into superior and inferior portions. Anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body.

Medical devices such as implants, trial components, provisional components, and the like are often grasped and/or manipulated with instruments or tools. Embodiments of the present disclosure may provide better grip of the medical device, more secure positioning of the medical device, and/or better access (for example, minimally invasive access) to a surgical location. The present disclosure shows and describes an interconnection, or interface, between an instrument and an implant. This description is made in the context of a spinal intervertebral spacer implant and a corresponding inserter instrument, the concepts embodied herein are applicable to other medical device/instrument interconnections.

FIG. 1 shows a spinal intervertebral spacer implant 10 connected to an instrument 12. The implant 10 and instrument 12 are securely connected so that there is little to no relative motion between the implant 10 and instrument 12, even as the implant 10 is forcibly introduced into an intervertebral space of a spine. The forcible introduction of the implant 10 into the intervertebral space may involve pushing, pulling, twisting, hammering, and/or other actions which subject the interconnection between the implant 10 and instrument 12 to substantial resultant forces. The interconnection between the implant 10 and instrument 12 is also readily releasable, so that little to no unintentional force is transmitted to the implant 10 upon disconnecting (or connecting) the instrument 12.

The instrument 12 includes a shaft portion 14, a handle portion 16 at one end of the shaft portion 14, and a knob 18 which may be located along either the shaft portion 14 or the handle portion 16. The knob 18 may be located in a middle portion of an overall length of the instrument 12, particularly in a location that is readily accessible to a user's fingers and thumb while grasping the handle portion 16. The knob 18 is set into the instrument 12 so as to be at least partially contained within the overall instrument profile.

FIG. 2 shows the knob 18 and handle portion 16 in greater detail. Knob 18 is configured to be rotated by a user. Such rotation may actuate a connection means at the end of shaft portion 14 which is configured to engage and disengage implant 10. It is noted that knob 18 may be located in different areas of instrument 12. For example, knob 18 may be placed at the base of handle portion 16. Further, knob 18 may be implemented as part of handle portion 16. For example, handle portion 16 may be implemented as two parts where the separate parts of the handle are configured to be rotated with respect to each other (e.g. at a connection point such as point 17) and that rotation is translated to the connection means.

FIGS. 3A-B show additional detail of the connection between the shaft portion 14 and the knob 18. FIG. 3B is a partial cross sectional view through the knob 18. A sleeve 20 is fixed within the knob 18 by a threaded connection and a snap ring 22. The sleeve 20 includes a noncircular central bore 24, which in this example is a hexagonal bore. A central shaft 26 of the shaft portion 14 is slidingly received within the central bore 24, and includes a noncircular boss 28 which is complementary to the central bore 24. Thus, in this example, the boss 28 is a hexagonal boss 28 which is sized for clearance in the central bore 24. The central bore 24 and boss 28 may be said to have a sliding interface.

When the knob 18 rotates, torque is transmitted to the central shaft 26, yet the central shaft 26 is free to slide axially with respect to the sleeve 20 and knob 18.

FIG. 4 shows that the central shaft 26 protrudes from an end face 32 of a working portion 30 of the shaft portion 14. When the implant 10 is held against the end face 32 while the knob 18 rotates in a first direction (such as clockwise), a threaded tip 34 of the central shaft 26 engages a corresponding threaded hole 36 in the implant 10 (FIGS. 5B, 7A). The sliding interface between the central bore 24 and boss 28 permits the threaded tip 34 to advance within the threaded hole 36 as the knob 18 is rotated in the first direction, and permits the threaded tip 34 to withdraw from the threaded hole 36 as the knob 18 is rotated opposite to the first direction. This threaded connection between the implant 10 and the instrument 12 may be referred to as a primary connection, at least because it has been described herein first.

FIG. 4 also shows that the working portion 30 of the shaft portion 14 includes a pair of fingers 38, 40 which also protrude from the end face 32. While the example of FIG. 4 shows two fingers arranged on opposite sides of the central shaft 26, other examples of this technology may have one finger, or more than two fingers. The fingers may be arranged around the central shaft 26 in a regular or irregular manner. The fingers may also be described as jaws or pincers.

Each finger moves in coordination with the axial motion of the central shaft 26. In the illustrated example, as the central shaft 26 advances within the implant 10, each finger pivots toward the central shaft 26. Conversely, as the central shaft withdraws from the implant 10, each finger pivots away from the central shaft 26. The fingers are biased to stand away from the central shaft 26. In another example, as the central shaft advances within the implant, each finger pivots away from the central shaft 26. In yet other examples, the motion of the fingers may be in a direction unrelated to the central shaft 26, although still coordinated with its motion.

Thus, the instrument 12 may be described as having at least two states: an open state (FIGS. 4, 5B, 8) and a closed, or locked, state (FIGS. 6B, 9). In the open state, the central shaft 26 is withdrawn into the instrument 12, although a leading portion of the threaded tip 34 protrudes from the end face 32. The open state is also characterized in that the fingers 38, 40 are maximally pivoted away from the central shaft 26. The fingers 38, 40 may be parallel in the open state. In the closed, or locked state, more of the threaded tip 34 protrudes from the end face 32 than in the open state. The locked state is also characterized in that the fingers 38, 40 are maximally pivoted toward the central shaft 26. The fingers 38, 40 also converge as they extend from the end face 32 in the locked state.

Referring to FIGS. 5B and 6B, the fingers 38, 40 are received in pockets 42, 44 in the implant 10. Since the example instrument 12 has two fingers 38, 40 arranged on opposite sides of the central shaft 26, the example implant 10 has two pockets 42, 44 arranged on opposite sides of the threaded hole 36. Other implant examples may have any number of pockets arranged to correspond with the fingers provided on the matching instrument. An implant may also have more pockets than there are fingers on the instrument.

As the threaded tip 34 initially advances within the threaded hole 36, the fingers 38, 40 slide into the corresponding pockets 42, 44 respectively, as shown in FIG. 5B. As the threaded tip 34 advances farther within the threaded hole 36, the fingers 38, 40 pivot toward the central shaft 26, as shown in FIG. 6B. Referring to FIG. 6B, the pockets 42, 44 converge as they extend into the implant 10. Each pocket may extend at an angle 64 with respect to a center longitudinal axis 62 of the threaded hole 36. The end of the implant 10 may be referred to as an instrument-facing surface 60. Any implant surface bearing a threaded hole and finger pocket may be an instrument-facing surface. While angles between zero degrees and 90 degrees are contemplated, angles between 10 and 80 degrees are preferred, and the example shows an angle of 45 degrees. The fingers 38, 40 also converge as they extend from the end face 32 in the locked state. Thus each finger 38, 40 may be said to engage an undercut geometry in the implant 10 when connected to the implant 10 in the locked state. The fingers 38, 40 may also be said to penetrate the implant at an angle with respect to a central longitudinal axis of the central shaft 26 or threaded hole 36. This arrangement directs the gripping force of the fingers 38, 40 against a pocket surface that resists forces tending to pull the implant 10 straight off of the instrument 12. As part of the rigidity of the connection, the fingers 38, 40 also urge the instrument-facing surface 60 of the implant 10 toward the end face 32 of the instrument 12. Tightening the threaded tip 34 controls the gripping force provided by the fingers 38, 40. Thus, the fingers 38, 40 act in conjunction with the threaded connection to actively retain the implant 10 on the instrument 12. The pinching or gripping connection between the implant 10 and the instrument 12 may be referred to as a secondary connection, at least because it has been described herein second.

One example of a mechanism for coordinating the movement of the fingers 38, 40 with the central shaft 26 is shown in FIGS. 5B and 6B. A collar 46 is coupled to the central shaft 26 by a retaining ring 48 so that the collar 46 slides axially along with the central shaft 26. Finger 38 is hinged to the collar 46 by a pin 50, and is hinged to the working portion 30 by a pin 52. Finger 40 is hinged to the collar 46 by a pin 54, and is hinged to the working portion 30 by a pin 56.

One example of a mechanism for biasing the fingers 38, 40 toward the open state is shown in FIGS. 5B and 6B. A spring 58 is installed between the central shaft 26 and the working portion 30. The spring 58 urges the central shaft 26 farther into the instrument 12, toward the open state.

While the preceding description has focused on one example, other examples are contemplated. One example may lack the threaded connection, and may have a lever-actuated finger gripping action.

In use, this interconnection may be quite similar to a plain threaded connection. However, the secondary connection of the gripping fingers 38, 40 improves instrument function by rotationally aligning the instrument 12 to the implant 10 and distributing service loads between the threaded connection and the gripping connection. Furthermore, as soon as the threaded connection is started, the rest of the locking operation may be performed one-handed.

The components disclosed herein may be made from metals, polymers, ceramics, glasses, composite materials, biological materials or tissues, insulators, conductors, semiconductors, or other biocompatible or non-biocompatible materials. Different materials may be used for individual components. Different materials may be combined in a single component.

It should be understood that the present system, kits, apparatuses, and methods are not intended to be limited to the particular forms disclosed. Rather, they are to cover all combinations, modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

In the foregoing Detailed Description, various features are grouped together in several embodiments for the purpose of streamlining the disclosure. This method of disclosure is not to be interpreted as reflecting an intention that the embodiments of the invention require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

Although the embodiments of the present disclosure and their advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A system comprising:
an implant comprising an instrument-facing surface, a threaded hole, and a pocket beside the threaded hole, wherein the threaded hole comprises a center longitudinal axis, wherein the threaded hole and the pocket extend into the implant through the instrument-facing surface, wherein the pocket extends into the implant at an angle with respect to the center longitudinal axis of the threaded hole; and
an instrument for connection to the implant, the instrument comprising an end face, a threaded rod, and a finger beside the threaded rod, wherein the threaded rod and the finger protrude through the end face;
wherein, when the instrument is connected to the implant, the instrument-facing surface abuts the end face, the threaded rod engages the threaded hole, and the finger extends into the implant pocket at the angle, wherein the finger urges the instrument-facing surface toward the end face.

2. The system of claim 1, wherein the pocket converges toward the center longitudinal axis of the threaded hole as the pocket extends into the implant.

3. The system of claim 1, wherein the implant is a spinal intervertebral spacer implant.

4. The system of claim 1, wherein the instrument is configured to receive force which causes the threaded rod to disengage with the threaded hole.

5. The system of claim 4, wherein when disengaging the threaded rod, the finger of the instrument is configured to withdraw from the pocket.

6. The system of claim 1, wherein the implant comprises a plurality of pockets which extend into the implant at an angle with respect to the center longitudinal axis of the threaded hole.

7. The system of claim 1, wherein the instrument comprises a plurality of fingers which correspond to the plurality of pockets which are configured to extend into the implant pockets at the angle, wherein the plurality of fingers are further configured to urge the instrument-facing surface toward the end face.

8. An apparatus for inserting an implant, the apparatus comprising:
an elongated instrument having a first end and a second end, the first end comprising a handle portion which includes a knob, the second end comprising an end face having a primary and secondary attachment means configured to attach to the implant,
wherein the instrument is configured such that actuation of the knob of the handle portion transmits force to the primary attachment means in order to engage and disengage the implant with the instrument, and wherein the secondary attachment means is configured to extend into the implant at an angle with respect to the center longitudinal axis of the primary attachment means.

9. The apparatus of claim 8 wherein the secondary attachment means is configured to move in axial coordination with the primary attachment means when engaging and disengaging the implant.

10. The apparatus of claim 9 wherein the instrument comprises a collar coupled to a central shaft that is configured to slide axially along the central shaft and wherein the secondary attachment means is pivotably coupled to the collar.

11. The apparatus of claim 8 wherein the attachment means comprises at least one finger configured to grip an inner surface of the implant.

12. The apparatus of claim 11 wherein the attachment means comprises a plurality of said fingers.

14. The apparatus of claim 8 wherein the angle of extension of the secondary attachment means is between 10 and 80 degrees.

15. The apparatus of claim 8 wherein the secondary attachment means is configured to prevent rotational misalignment forces between the implant and the instrument.

16. The apparatus of claim 8 wherein the primary attachment means comprises a threaded rod configured to thread within the implant.

17. An implant apparatus comprising:
a contact surface configured to attach and release from an insertion tool, said contact surface defining a primary attachment aperture and at least one secondary attachment aperture,
wherein the primary aperture is threaded and configured to receive a corresponding threaded shaft from the insertion tool,
wherein the at least one secondary aperture extends within the implant at an angle with respect to the primary aperture and the secondary aperture is configured to receive a corresponding gripping finger of the insertion tool.

18. The implant apparatus of claim 17 wherein the at least one secondary attachment aperture includes a plurality of apertures disposed on opposing sides of the primary aperture.

19. The implant apparatus of claim 18 wherein the plurality of apertures are angled with respect to the central axis of the primary aperture.

20. A method for inserting an implant device, said method comprising:
attaching an implant device to an insertion instrument, wherein the implant device comprises an instrument-facing surface, a threaded hole, and a pocket beside the threaded hole, wherein the threaded hole comprises a center longitudinal axis, wherein the threaded hole and the pocket extend into the implant through the instrument-facing surface, wherein the pocket extends into the implant at an angle with respect to the center longitudinal axis of the threaded hole, wherein the instrument comprises an end face, a threaded rod, and a finger beside the threaded rod, wherein the threaded rod and the finger protrude through the end face and wherein, when the instrument is connected to the implant, the instrument-facing surface abuts the end face, the threaded rod engages the threaded hole, and the finger extends into the implant pocket at the angle;
inserting the implant device into a subject; and
releasing the implant device from the insertion instrument, wherein said releasing comprises unthreading the threaded rod from the threaded hole wherein unthreading the threaded rod also axially removes the finger from the pocket of the implant device.
